(19) United States
(12) Patent Application Publication
(10) Pub. No.: US 2002/0156407 A1
(43) Pub. Date: Oct. 24, 2002

(54) Ambidextrous arm support vest

(76) Inventor: Alberta Sue Estep, Mendenhall, MS (US)

Correspondence Address: R. Bennett Ford, Jr., 2355 Drusilla Lane, Baton Rouge, LA, 70809, US

(21) Appl. No.: 10/165,720

(22) Filed: Jun. 7, 2002

Related U.S. Application Data
(63) Continuation-in-part of application No. 09/737,285, filed on Dec. 4, 2000.

(51) Int. Cl. A61F 5/00

(52) U.S. Cl. 602/4

(57) ABSTRACT

A garment for supporting the disabled or paralyzed arm of a wearer. The garment has a fabric member with an open bottom, a head opening in its top, and a pair of arm holes, which allow it to be worn like a standard shirt or vest. An arm support pocket is positioned on the front of the garment. The arm support pocket is a sleeve in which the disabled arm is placed to be retained and supported. A utility pocket is also preferably positioned near the bottom of the garment. The utility pocket is preferably divided to keep items in the pocket from migrating to locations where they may be difficult to retrieve. The garment may also have an openable and closeable seam in the back to facilitate dressing and undressing by the wearer.

### HISTORY

This is a continuation-in-part of U.S. patent application Ser. No. 09/737,285, now abandoned, which is hereby incorporated by reference in its entirety.

### BACKGROUND OF THE INVENTION

1. Field of the Invention

This invention relates to limb slings in general and to slings for patients with paralyzed arms in particular.

2. Prior Art

Patients with paralyzed arms, such as recovering stroke victims and patients with nerve damage affecting an arm, require support and restraint for the arm. If the limb is unrestrained, it will swing as the patient moves. The arm may brush against objects that are sharp, hot, or otherwise dangerous to the patient. In a patient with damage to his nervous system, there frequently will not be any sense of touch to warn the patient of contact with the dangerous object. This can lead to serious bums or other injury to the patient. Therefore, the patient's arm needs to be restrained to minimize the chances that it will come into contact with such objects.

The paralyzed arm also needs to be supported. In many patients with paralysis in a limb, the muscles in the limb will atrophy from disuse. Additionally, the muscles in the affected limb are unable to be stimulated to varying degrees because of the paralysis. The result of these two conditions is that the limb is supported solely by the joint connecting it to the trunk of the body. In the case of a paralyzed arm, this joint is the shoulder blade, which has no bone connection to the skeleton. The weight of an arm literally hanging from the shoulder can cause the shoulder blade to become dislocated and can lead to further complications for the patient. Therefore, the weight of the paralyzed limb needs to be supported to reduce the strain on the shoulder.

Although a paralyzed arm needs to be supported and restrained, both tasks need to be performed without placing excessive constraint on the arm. The restraining and supporting device will likely be worn by the patient day after day, in many cases for the rest of the patient's life. If the device is too tight, pressure or “bed” sores may develop, again causing additional health problems for the patient.

Patients suffering from paralysis in an arm are obviously beset with a disability. Such patients have a desire and need to live their lives as independently as possible. Therefore, if at all possible, any garment that they wear regularly should be something they can put on without assistance. Moreover, if the garment is to engage the paralyzed limb, they should be able to secure the paralyzed limb in the garment without assistance as well.

Patients with a paralyzed arm may have difficulty retrieving items from pockets. For example, a patient with a paralyzed right arm would have considerable difficulty retrieving an object from the left inside pocket of jacket. Obviously, such a patient may avoid this difficulty by not placing anything in his left inside pocket. However, with certain garments, items can migrate within a pocket, and items placed in one place may not still be in a spot convenient for retrieval at a later time.

It will be more efficient if the same garment can achieve all of the foregoing goals for patients suffering from paralysis on either side of the body. Therefore, a garment that can be worn with equal ease by persons suffering from right side or left side paralysis would be advantageous. No prior art device is known to achieve all of these goals. Therefore, an arm support garment meeting the following objectives is desired.

### OBJECTS OF THE INVENTION

It is an object of the invention to provide a garment that can restrain a paralyzed arm, and thereby lessen the risk of injury to the arm.

It is another object of the invention to provide a garment that can support a paralyzed arm and thereby reduce the likelihood of injury to the shoulder blade.

It is still another object of the invention to provide a garment that can support and restrain a paralyzed arm in a sufficiently loose manner to substantially avoid the risk of creating pressure sores.

It is yet another object of the invention to provide a garment for supporting and restraining a paralyzed arm which the patient can put on without any assistance.

It is still another object of the invention to provide a garment for supporting and restraining a paralyzed arm which can be worn with equal convenience by patients with left arm or right arm paralysis.

It is still another object of the invention to provide a garment for supporting and restraining a paralyzed arm which is configured to retain objects in a convenient position for retrieval by the patient.

It is still another object of the invention to provide a garment for supporting and restraining a paralyzed arm that is adjustable to fit patients of different body sizes.

It is still another object of the invention to provide a garment for supporting and restraining a paralyzed arm that is washable.

### SUMMARY OF THE INVENTION

The preferred embodiment of the invention is a generally cylindrical upper body garment. The garment has a top and a bottom. The bottom is open to pass over the head of the wearer and also sized to comfortably encircle the trunk of the wearer. The top of the garment contains an opening sized to pass over the wearer's head and rest upon his shoulders as in a standard shirt or vest. The sides of the garment contain a pair of aligned arm openings sized to allow the wearer's arms to extend outside of the garment. The arm openings may or may not be provided with sleeves. In one embodiment, the rear of the garment is split from top to bottom and provided with Velcro type fasteners to hold the rear of the garment closed. In this embodiment, the garment may be pulled over the wearer's head like a T-shirt or slipped on one arm at a time and then closed in the back.

The front of the garment is provided with an arm support pocket. In the preferred embodiment, the arm support pocket comprises a piece of fabric affixed at its upper and lower edges to the front of the garment. The arm support pocket also has two open ends, generally opposite each other and generally perpendicularly positioned relative to the open top and bottom of the garment so that the length of the arm support pocket extending between the two open ends is generally horizontal. The arm support pocket is preferably positioned on the front of the garment so that it will lie across the sternum of the wearer when the garment is in place.

The wearer will place his good arm through one open end of the arm support pocket and out the opposite end. He will grab his arm that requires support and pull it back through the arm support pocket with his good arm, leaving the paralyzed arm supported and restrained by the arm support pocket. As should be apparent, the garment may be used with equal convenience by wearers suffering disability in either the left or the right arm.

In one preferred embodiment, the arm support pocket is located on the inside of the garment. In this embodiment, the front of the garment is provided with a pair of openings in alignment with the open ends of the arm support pocket. The openings will provide passage to the arm support pocket from the exterior of the garment.

The preferred embodiment of the invention is also provided with a partitioned pocket. In the preferred embodiment, the partitioned pocket comprises a swath of fabric extending over the bulk of the front of the garment. The swath is secured at its bottom and at both sides, but is open at the top. Significantly, it is provided with a vertical partition near its midpoint. This will separate the pocket into a left and right section. By partitioning the pocket, the wearer can be assured that when he places an object in the pocket, the object will not migrate to a portion of the pocket that is difficult for him to access with his unrestrained healthy arm. By having two such pockets, the garment remains equally useful to wearers suffering a disability in either arm.

### BRIEF DESCRIPTION OF THE DRAWINGS

FIG. 1 is front view of a preferred embodiment of the invention having an open back with Velcro® type fasteners and a partitioned pocket.

FIG. 2 is a perspective view of the embodiment of the invention shown in FIG. 1 in place on a wearer with a disabled right arm and with the back open for illustration.

FIG. 3 is a perspective partial cut-away view of the embodiment of the invention shown in FIG. 1 in place on a wearer with a disabled right arm.

FIG. 4 is a perspective partial cut-away view of the embodiment of the invention shown in FIG. 1 in place on a wearer with a disabled left arm.

FIG. 5 is a perspective view of another preferred embodiment of the invention wherein the arm support pocket is located on the inside of the garment.

FIG. 6 is a perspective view of the arm support pocket.

FIG. 7 is a perspective view of the preferred embodiment of the invention illustrated in FIG. 5 in place on a wearer with a disabled right arm.

FIG. 8 is a side view of the preferred embodiment of the invention illustrated in FIG. 5 in place on a wearer with a disabled fight arm.

FIG. 9 is a perspective view of the preferred embodiment of the invention illustrated in FIG. 5 in place on a wearer with a disabled right arm.

FIG. 10 is a front view of a preferred embodiment of the invention having a utility pocket located on the outer surface of the arm support pocket.

FIG. 11 is a side view of a preferred embodiment of the invention having a plurality of gathering strips.

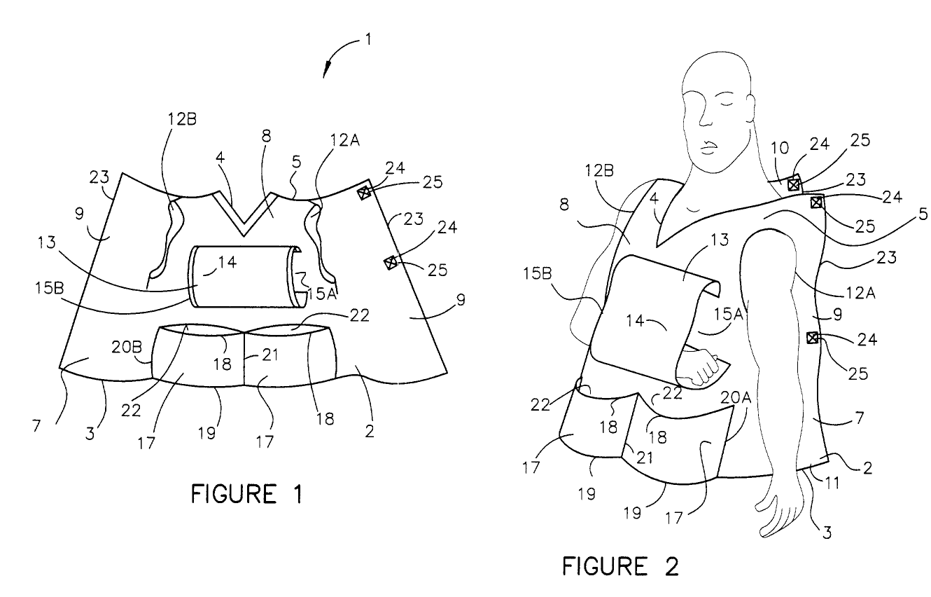

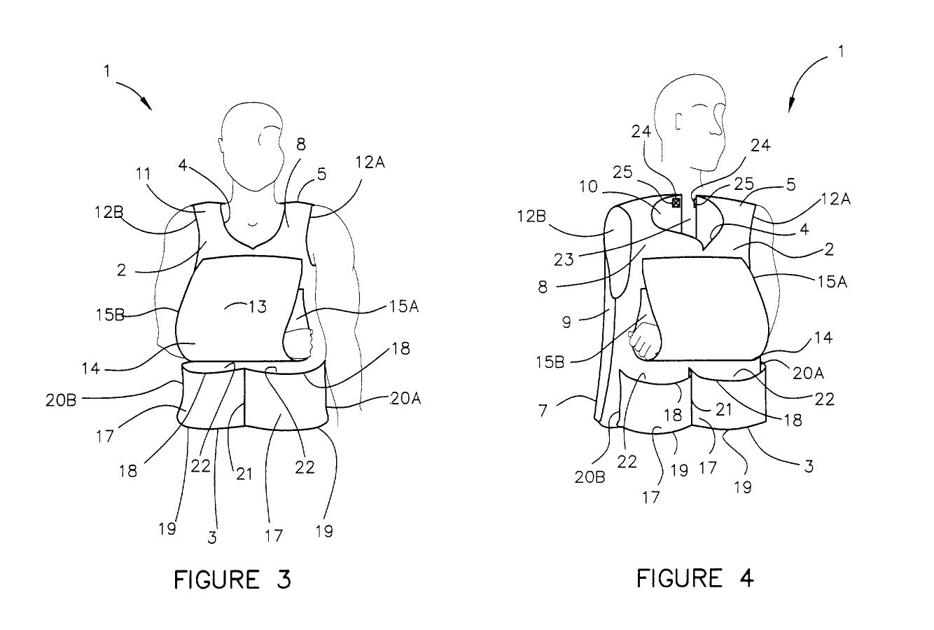

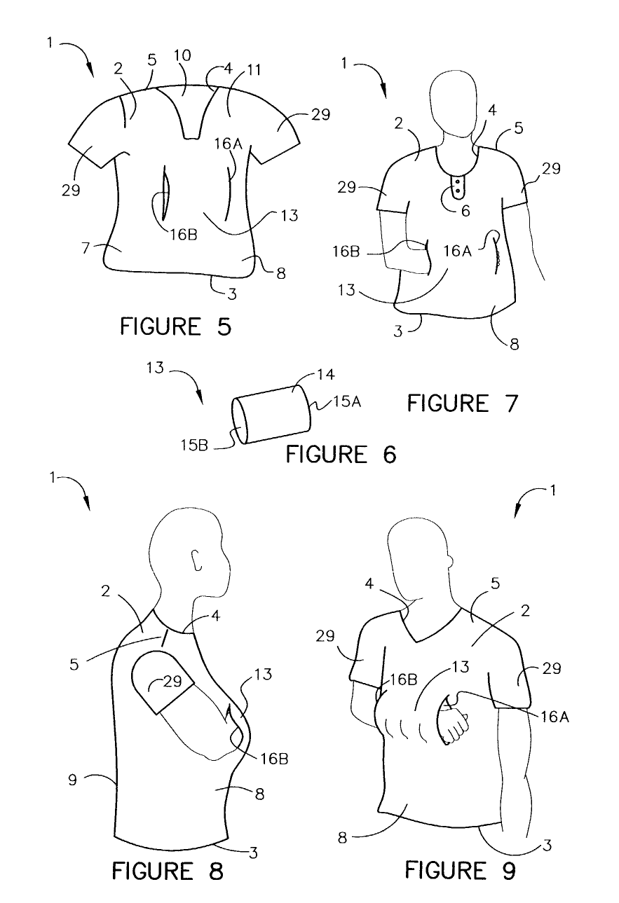

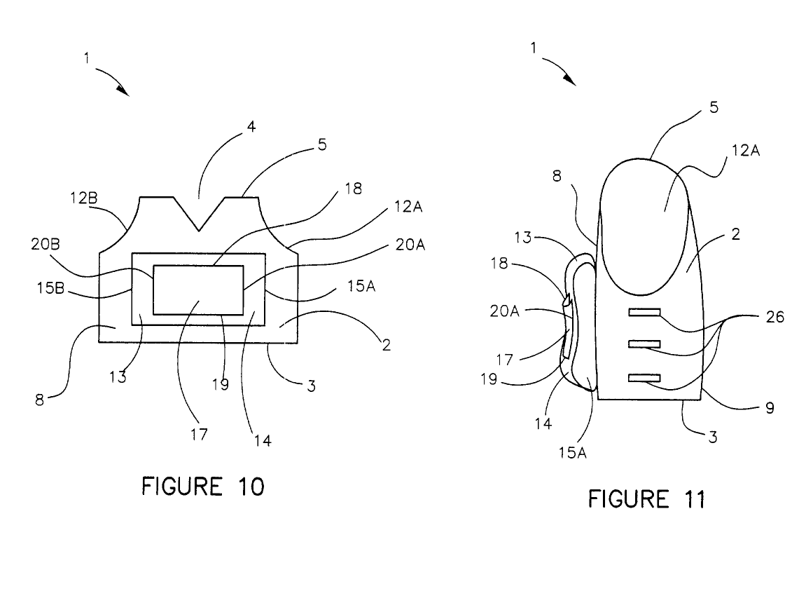

### DETAILED DESCRIPTION OF THE PREFERRED EMBODIMENT

The preferred embodiment of the invention is a garment 1 for supporting and restraining a patient's injured or paralyzed arm. Garment 1 comprises a generally cylindrical fabric member 2. Fabric member 2 is sized to encircle the trunk of the patient and to extend from about the wearer's neck to at least about the wearer's waist, although fabric member 2 could be longer if desired. Garment 1 is intended to worn in the same fashion as a standard shirt or vest. As such, fabric member 2 has an open bottom 3 and a head opening 4 in its top 5. Head opening 4 should be sized to allow the passage of the wearer's head. Head opening 4 may be provided with one or more buttons 6, or other fasteners, in order to increase or decrease the size of head opening 4.

Fabric member 2 is, as its name implies, preferably made of a fabric 7 such as cotton, polyester, cotton-polyester blends (most preferably, a 65/35 polyester/cotton blend), or other standard clothing materials. Fabric 7 should preferably be machine washable. In fabric member 2, fabric 7 extends between top 5 and bottom 3. Fabric member 2 has a front side 8 and back side 9. Fabric member 2 also has an inner surface 10 and an outer surface 11.

Fabric member 2 is further provided with a pair of arm openings 12A and 12B positioned to align with the arms of the wearer. Arm openings 12A and 12B allow the wearer's arms to extend out of fabric member 2. Arm openings 12A and 12B may be provided with short or long sleeves 29 if desired.

Garment 1 is also preferably provided with an arm support pocket 13. In one preferred embodiment, arm support pocket 13 is attached to fabric member 2. In a preferred embodiment, arm support pocket is a hollow fabric sleeve 14 comprising a single rectangular swath of fabric sewn at its upper and lower edges to the outer surface 11 of the front side 8 of fabric member 2. The sides of the swath perpendicular to its upper and lower edges will be left unattached, creating a pair of opposite open ends 15A and 15B to sleeve 14. Both open ends 15A and 15B should be of sufficient size to permit passage of the wearer's hands. In the preferred embodiment, the swath of fabric sewn to fabric member 2 to form arm support pocket 13 measures about fourteen inches in width and about seventeen inches in height, width being that dimension parallel to the shoulder line of the wearer when garment 1 is in place. Although the preferred swath is about seventeen inches in height, its upper and lower edges are sewn to fabric member 2 along generally horizontal lines that are preferably only about nine inches apart. This will allow the swath, together with the portion of front side 8 of fabric member 2 which the swath overlays, to form sleeve 14 rather than simply forming a double layered garment. Although sleeve 14 has been described as being formed by attaching a swath of fabric to front side 8 of fabric member 2 so that fabric member 2 forms part of sleeve 14, sleeve 14 could also be formed entirely from a separate swath or swaths of fabric and then attached to fabric member 2.

In another preferred embodiment, arm support pocket 13 is located on the inner surface 10 of fabric member 2. In this embodiment, sleeve 14 may be formed in the manner described above, except that rather than attaching the swath to the outer surface 11 of fabric member 2, the swath is sewn to the inner surface 10. Alternatively, a fully formed sleeve 14 may be prepared and attached the inner surface 10. In either case, the wearer should preferably have a way to get his arm back inside garment 1 and into sleeve 14. Passage back to the interior of garment 1 is provided by a pair of arm support pocket access apertures 16A and 16B. Arm support pocket access apertures 16A and 16B should be sized to allow passage of the wearer's hand and arm and should be positioned immediately outside open ends 15A and 15B so that a wearer could place his or her hand through either aperture 16A or 16B and immediately into the corresponding open end 15A or 15B of sleeve 14.

The purpose of arm support pocket 13 is to support and restrain the wearer's arm. By attaching sleeve 14 to fabric member 2, the weight of the patient's arm can be shifted from the patient's shoulder blade to the patient's shoulders by virtue of the fact that garment 1, like any shirt or vest, hangs over the wearer's shoulders. Having the weight of the patient's arm transferred to his shoulders is particularly desirable when the wearer is suffering from paralysis in an arm. In such a case, the muscles, tendons, and ligaments in the wearer's shoulder blade are likely to atrophy from disuse. As they weaken over time, the shoulder blade can become incapable of supporting the weight of the arm, and can become dislocated from the weight of the paralyzed limb hanging from it. By transferring the weight of the arm to the shoulders, garment 1 makes it possible for the weight of the wearer's arm to rest on his collarbones which have a direct bony connection to the rest of the skeleton, unlike the shoulder blade, and thus will not be as significantly affected by paralysis induced atrophy of the surrounding muscles and connecting tissues. Garment 1 will also distribute some of the weight of the arm to the other side of the wearer's body which would typically not be suffering from paralysis and, thus, would be much more capable of supporting the weight of the paralyzed arm.

Whichever embodiment of arm support pocket 13 is used, it should preferably be sized and positioned to hold the wearer's arm across his trunk at about his sternum. By restraining the wearer's arm in this location, garment 1 will reduce the likelihood of the wearer's (paralyzed) arm coming into contact with a harmful object without the wearer knowing about it.

Sleeve 14 should be strong enough to support the weight of the wearer's arm, but should preferably not be tight on the wearer's arm. If the wearer's arm were tightly bound for an extended period of time, pressure sores might result. Therefore, a loose fit of sleeve 14 is desired.

Garment 1 may also be provided with a utility pocket 17. In the preferred embodiment, utility pocket 17 is positioned on the front side 8 of fabric member 2 below arm support pocket 13. Utility pocket 17 preferably extends substantially the full width of the front side 8 of fabric member 2. Utility pocket 17 has an open top 18, and closed bottom 19 and sides 20A and 20B. One or more vertical dividers 21 are preferably positioned between sides 20A and 20B. Utility pocket 17 is preferably formed by sewing a swath of fabric to fabric member 2 at bottom 19 and sides 20A and 20B of utility pocket 17. Dividers 21 are preferably formed by sewing a portion of the swath of fabric to fabric member 2 along a generally vertical line extending from bottom 19 to top 18 between sides 20A and 20B, thereby segregating utility pocket 17 into one or more sections 22. In the preferred embodiment, a single divider 21 is placed at approximately the midpoint between sides 20A and 20B, thereby separating utility pocket 17 into two sections 22 of approximately the same size.

A utility pocket 17 as described above is particularly useful to patients suffering from paralysis in one arm or who otherwise cannot use one arm. The wearer can place an item in utility pocket 17. Because of divider 21, the item cannot migrate to the opposite side of utility pocket 17, where it might be difficult for the wearer to reach—similar to the difficulty one might encounter retrieving keys from one's left pocket with one's right hand. Thus, because of divider 21, the wearer knows that he will be able to easily retrieve items placed in utility pocket 17. Also, in the preferred embodiment, this is equally true regardless of which of the patient's arms is paralyzed because there is a section 22 on both sides of garment 1.

Although utility pocket 17 has been described as being positioned below sleeve 14, in another embodiment, utility pocket 17 may be included on the outer surface of sleeve 14. This can provide another convenient location for storing items. Additionally, a pocket in this location will be more accessible to both arms so that the wearer will be more likely to be able to reach items placed in pocket 17 regardless of which arm is his good arm. Placing utility pocket 17 on sleeve 14 may also reduce the need to divide pocket 17 into sections, although this certainly can be done if desired. Positioning utility pocket 17 on sleeve 14 will also be particularly desirable in embodiments of garment 1 designed to be relatively short in length, ending just below sleeve 14. Of course, in longer versions of garment 1 utility pocket 17 could be placed in either location, both, or omitted altogether depending upon designer preference.

In one preferred embodiment, there is a seam 23 in the back side 9 of fabric member 2. Seam 23 may extend the full length of fabric member 2 so that garment 1 may be laid out flat when seam 23 is open or seam 23 may extend only part of the length of fabric member 2 so that when seam 23 is open it only effectively increases the size of head opening 4. Seam 23 is provided with fastening devices 24 such as Velcro® strips 25 on each side of seam 23 or other conventional fastening devices 24 such as buttons, snaps, zippers, etc. However, a Velcro® type fastener is preferred because of the ease with which such fastening devices 24 may be opened and closed.

In one preferred embodiment, garment 1 is configured to be adjustable in size. This is preferably accomplished by providing garment 1 with one or more gathering strips 26, preferably about four inches in length each. Gathering strips 26 are preferably strips of Velcro® type fastening devices with hooks on one end and loops on the other, each making up approximately half the length of gathering strip 26. Gathering strips 26 are preferably attached to garment 1 along their length or at least at each end and positioned on garment 1 so that they will be positioned generally parallel to the waist of the wearer. By folding each strip 26 so that the hook end is attached to the loop end, gathering strips 26 will gather the material of garment 1 and thereby reduce its effective circumference. Thus, an extra-large sized garment 1 could be reduced to a large by folding gathering strips 26 so that their ends are connected. Similarly, a medium could be reduced to a small in the same fashion. The preferred location for gathering strips 26 is on the side of garment 1 below the arms. The preferred number of gathering strips 26 is three per side, although a greater or smaller number can be used if desired. Additionally, gathering strips 26 could be placed on only one side of garment 1 or in different locations beside the side or garment 1. Gathering strips 26 could also be rotated ninety degrees so that they were positioned generally parallel to the spine of the wearer. In this position, gathering strips 26 could be used to vary the length of garment 1 rather than its circumference. Although gathering strips 26 have been described as comprising strips of Velcro®, they could utilize any other conventional type of fastening device, such as snaps, buttons, or hooks to connect one end of strip 26 to the other.

Seam 23 can facilitate the ability of a disabled wearer to put garment 1 on alone. By opening seam 23, the wearer can make it easier to get garment 23 over his head. Where seam 23 will allow garment 1 to be separated completely in the back, seam 23 can allow a wearer to avoid having to pull garment 1 over his head at all. In this embodiment, garment 1 can be placed over the disabled arm first, then installed over the healthy arm, and finally secured in the back by closing seam 23, preferably by placing the Velcro® strips 25 into contact with each other. Alternatively, the wearer could position garment 1 over his disabled arm and then pull garment 1 over his head and into place with his healthy arm.

Once garment 1 is in place, the wearer will take his healthy arm and pass it though arm support pocket 13 and grab his disabled arm, preferably at the hand or wrist. He will then pull his disabled arm into arm support pocket 13 as he withdraws his healthy arm. This will leave the disabled arm supported by and restrained in arm support pocket 13. This will work the same way whether arm support pocket 13 is on the inside or outside of fabric member 2. Also, as should be apparent, regardless of which arm is disabled the wearer will be able to secure it in arm support pocket 13 with his opposite healthy arm using the present invention.

Other uses and embodiments of the invention will occur to those skilled in the art from the foregoing disclosure and figures, and are intended to be included within the scope and spirit of the following claims.

I claim:

1. A garment for supporting and restraining a disabled left or right arm of a human wearer, said wearer having a head, a neck, arms, hands, shoulders, a trunk, a sternum, and a waist, said garment comprising:
a generally cylindrical fabric member having a front side and a back side, said fabric member configured to extend downward from the neck of said wearer and sized to encircle the trunk of said wearer, said fabric member having a top and an open bottom and fabric extending between said top and said bottom, said top containing a head opening sized to allow passage of said wearer's head, said fabric member further comprising a pair of arm openings positioned to correspond with said arms of said wearer and sized to allow passage of said arms, and
an arm support pocket comprising a generally horizontally positioned hollow fabric sleeve attached to the front side of said fabric member, said arm support pocket having two opposite open ends, said open ends and said fabric sleeve sized to allow a wearer to pass his hand into one open end of the arm support pocket, through the fabric sleeve, and out the opposite open end, said fabric sleeve being further configured to support the arm of a wearer from the wearer's shoulders and to simultaneously restrain the arm adjacent to the wearer's trunk when the wearer's arm is placed in said sleeve.

2. A garment for supporting and restraining a disabled left or right arm of a human wearer according to claim 1 further comprising a utility pocket positioned on said front side of said fabric member, said utility pocket extending substantially the full width of said front side of said fabric member, said pocket having an open top, a closed bottom, closed sides, and at least one vertical divider positioned between said closed sides, said divider configured to separate said pocket into a plurality of sections whereby items placed in one section may be prevented from migrating into another section.

3. A garment for supporting and restraining a disabled left or right arm of a human wearer according to claim 2 wherein said arm support pocket is positioned to support an arm of the wearer positioned in said support pocket at about the wearer's sternum.

4. A garment for supporting and restraining a disabled left or right arm of a human wearer according to claim 3 wherein the back side of said fabric member contains a seam configured to be opened and closed.

5. A garment for supporting and restraining a disabled left or right arm of a human wearer according to claim 4 wherein said seam extends from the top of said fabric member to the bottom of said fabric member.

6. A garment for supporting and restraining a disabled left or right arm of a human wearer according to claim 5 wherein said seam is provided with Velcro® type fastening devices for opening and closing said seam.

7. A garment for supporting and restraining a disabled left or right arm of a human wearer according to claim 1 wherein said fabric member has an inner surface and an outer surface.

8. A garment for supporting and restraining a disabled left or right arm of a human wearer according to claim 7 wherein said arm support pocket is mounted on said inner surface of said fabric member and wherein front side of said fabric member contains a pair of arm support pocket access apertures positioned to allow a wearer to place his hand through either access aperture and immediately into said arm support pocket.

9. A garment for supporting and restraining a disabled left or right arm of a human wearer according to claim 8 further comprising a utility pocket positioned on said front side of said fabric member, said utility pocket extending substantially the full width of said front side of said fabric member, said pocket having an open top, a closed bottom, closed sides, and at least one vertical divider positioned between said closed sides, said divider configured to separate said pocket into a plurality of sections whereby items placed in one section may be prevented from migrating into another section.

10. A garment for supporting and restraining a disabled left or right arm of a human wearer according to claim 9 wherein said arm support pocket is positioned to support an arm of the wearer positioned in said support pocket at about the wearer's sternum.

11. A garment for supporting and restraining a disabled left or right arm of a human wearer according to claim 10 wherein the back side of said fabric member contains a seam configured to be opened and closed.

12. A garment for supporting and restraining a disabled left or right arm of a human wearer according to claim 11 wherein said seam extends from the top of said fabric member to the bottom of said fabric member.

13. A garment for supporting and restraining a disabled left or right arm of a human wearer according to claim 12 wherein said seam is provided with Velcro® type fastening devices for opening and closing said seam.

14. A garment for supporting and restraining a disabled left or right arm of a human wearer according to claim 1 wherein said arm support pocket has an outer surface and further comprising a utility pocket positioned on said outer surface of said arm support pocket.

15. A garment for supporting and restraining a disabled left or right arm of a human wearer according to claim 1 wherein said garment is configured to be adjustable in size.

16. A garment for supporting and restraining a disabled left or right arm of a human wearer according to claim 15 wherein said garment is provided with at least one gathering strip.
